(19) United States
(12) Patent Application Publication
(10) Pub. No.: US 2018/0029959 A1
(43) Pub. Date: Feb. 1, 2018

(54) LEWIS ACID CATALYSTS FOR PRODUCING TOLUENE AND METHOD FOR MANUFACTURING TOLUENE USING THE SAME

(71) Applicant: KOREA INSTITUTE OF SCIENCE AND TECHNOLOGY, Seoul (KR)

(72) Inventors: Jungho JAE, Seoul (KR); Yanuar Philip WIJAYA, Seoul (KR); Dong Jin SUH, Seoul (KR); Hyun JOO LEE, Seoul (KR); Ivan KRISTIANTO, Seoul (KR)

(21) Appl. No.: 15/625,627

(22) Filed: Jun. 16, 2017

(30) Foreign Application Priority Data
Jul. 26, 2016 (KR) 10-2016-0095024

(51) Int. Cl. C07C 15/06; C07C 2/86; C07C 1/20

(52) U.S. Cl. CPC C07C 15/06; C07C 1/20; C07C 2/862; C07C 2521/08; C07C 2521/04; C07C 2529/08

(57) ABSTRACT

Disclosed is a Lewis acid catalyst for preparation of toluene from 2-methylfuran and a method for preparing toluene from 2-methylfuran by using the same. The catalyst is a zeolite catalyst ion-exchanged with a metal or a metal halide catalyst. The catalyst accelerates the cycloaddition of 2-methylfuran with ethylene and inhibits oligomerization as a side reaction, and thus allows production of toluene from 2-methylfuran with high yield and high selectivity.

### CROSS-REFERENCE TO RELATED APPLICATION

This application claims the priority to Korean Patent Application No. 10-2016-0095024, filed on Jul. 26, 2016, and all the benefits accruing therefrom under 35 U.S.C. §119, the contents of which in its entirety are herein incorporated by reference.

### BACKGROUND

### 1. Field

The present disclosure relates to a Lewis acid catalyst for producing toluene from 2-methylfuran and a method for preparing toluene from 2-methylfuran using the same.

### DESCRIPTION ABOUT NATIONAL SUPPORT RESEARCH AND DEVELOPMENT

This study is made by the support of Korea Ministry of Science, ICT and Future Planning under the supervision of Korea Institute of Science and Technology [Research management specialized agency: Nation Research Council of Science & Technology, research subject title: ‘Development of Advanced Fuel/Material Production Technology Based on Integrative Utilization of Lignocellulosic Biomass’ (Subject Identification No.: CAP-11-04-KIST)].

### 2. Description of the Related Art

Lignocellulose in wooden materials, herbal materials or the like includes cellulose, hemicellulose and lignin, and may be decomposed into monosaccharides, such as glucose or xylose, through a saccharification process. In the case of such monosaccharides, they may be converted into transport fuel, such as bio-ethanol. In addition, some technologies for converting them into high-added value compounds through a chemical process have been given many attentions recently. For example, in the case of glucose, it may be converted into 2,5-dimethylfuran through dehydration and hydrodeoxygenation.

2,5-dimethylfuran has a high octane value and energy density, and thus has been spotlighted as a next-generation transport fuel substituting for bio-ethanol. Recently, it has been reported that 2,5-dimethylfuran is converted into para-xylene used as a solvent for manufacture of paint and polymer products of polyester, polyethylene terephthalate (PET) and resin for packaging, through the cycloaddition with ethylene (U.S. Pat. No. 8,314,267B2). According to the related art, it is shown that commercially available catalysts having strong acid center property, such as H-Beta zeolite and tungstated zirconia, provide high activity (>80% yield) in the preparation of para-xylene (US20140296600A1). In addition, the present inventors have disclosed that a silica alumina aerogel catalyst having a large specific surface area and mesopores significantly increases a reaction rate through rapid mass transfer.

Similarly to the methods for preparing para-xylene from 2,5-dimethylfuran, xylose-derived 2-methylfuran may be converted into toluene through the cycloaddition reaction with ethylene. However, according to the related art, in the case of such a catalyst as zeolite having high activity in the preparation of para-xylene, it shows significantly low activity (<45% yield) in the preparation of toluene. The reason why 2-methylfuran shows low toluene selectivity is oligomerization occurring as a side reaction. Moreover, in the case of a strong acid catalyst, it is known that the catalyst accelerates such a side reaction. Therefore, it is required to develop a catalyst and reaction condition capable of increasing toluene selectivity, while inhibiting oligomerization, in order to increase the efficiency of the 2-methylfuran process.

### SUMMARY

The present disclosure is directed to providing a Lewis acid catalyst for use in the preparation of toluene from 2-methylfuran.

The present disclosure is also directed to providing a method for preparing toluene from 2-methylfuran by using the catalyst.

In one aspect, there is provided a catalyst for use in the preparation of toluene from 2-methylfuran, which is a Lewis acid catalyst and is a zeolite catalyst ion-exchanged with at least one metal.

According to an embodiment, the catalyst may be a zeolite catalyst ion-exchanged with at least one metal selected from the group consisting of alkali metals, transition metals and post-transition metals.

According to another embodiment, the catalyst may be a zeolite catalyst ion-exchanged with at least one alkali metal.

According to still another embodiment, the catalyst may be a zeolite catalyst ion-exchanged with Li or Na.

According to yet another embodiment, the catalyst may be a Y-zeolite catalyst having an FAU structure.

In another aspect, there is provided a catalyst for use in the preparation of toluene from 2-methylfuran, which is a Lewis acid catalyst and is a metal halide catalyst.

According to an embodiment, the catalyst may be a metal halide catalyst including: at least one cation selected from the group consisting of transition metals and post-transition metals; and a halogen anion.

According to another embodiment, the catalyst may be a metal chloride.

According to still another embodiment, the catalyst may be AlCl₃, VCl₃ or CuCl₂.

In still another aspect, there is provided a method for preparing toluene from 2-methylfuran by using the catalyst.

According to an embodiment, the method may include carrying out reaction of 2-methylfuran with ethylene in the presence of the catalyst and an organic solvent.

According to another embodiment, the reaction temperature and reaction pressure are 200-300° C. and an initial ethylene pressure of 20-37 bars, respectively.

According to still another embodiment, the reaction time may be 4-48 hours.

According to still another embodiment, the organic solvent may be a polar aprotic organic solvent.

According to yet another embodiment, the polar aprotic organic solvent may be at least one selected from the group consisting of 1,4-dioxane and tetrahydrofuran.

According to an aspect of the present disclosure, there is provided a Lewis acid catalyst for use in the preparation of toluene from 2-methylfuran.

According to another aspect of the present disclosure, there is provided a method for preparing toluene from 2-methylfuran by using the catalyst.

The catalyst for preparing toluene disclosed herein has properties as a Lewis acid catalyst, and thus accelerates the cycloaddition reaction between 2-methylfuran and ethylene, while inhibiting oligomerization as a side reaction. Thus, it is possible to obtain toluene from 2-methylfuran with high yield and high selectivity (>70%).

### BRIEF DESCRIPTION OF THE DRAWINGS

FIG. 1 shows the chemical molecular structures and reaction scheme for the final product and side products obtained through cycloaddition and dehydration from 2-methylfuran.

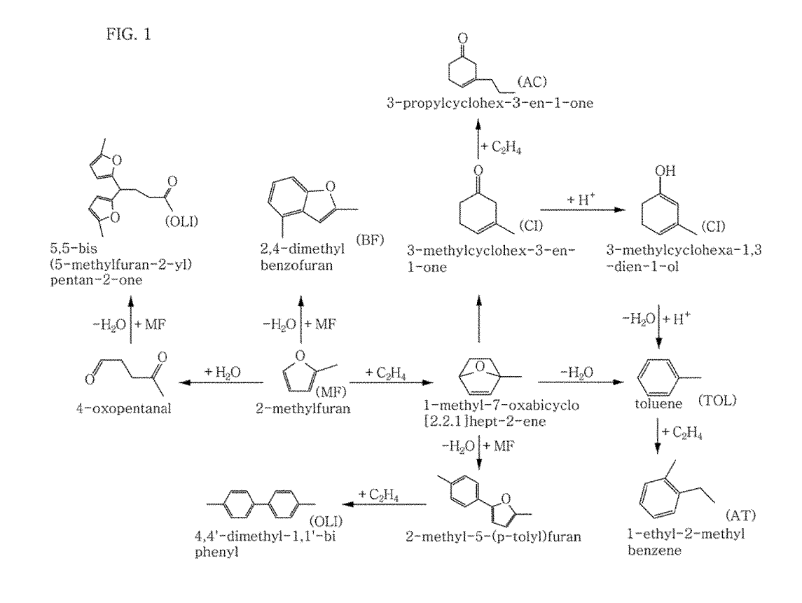

FIG. 2 is a graph illustrating the results of preparation of toluene from 2-methylfuran using the catalysts of Examples 1-6 according to an embodiment of the present disclosure.

FIG. 3 is a graph illustrating the results of preparation of toluene from 2-methylfuran using the catalyst of Example 2 according to an embodiment of the present disclosure.

### DETAILED DESCRIPTION

Hereinafter, the present disclosure will be explained in more detail.

In one aspect, there is provided a catalyst for use in the preparation of toluene from 2-methylfuran derived from biomass, which is a Lewis acid catalyst.

According to an embodiment, the catalyst may be a metal ion-exchanged zeolite catalyst.

According to another embodiment, the metal may be at least one metal selected from the group consisting of alkali metals, transition metals and post-transition metals, and particularly an alkali metal in view of the preparation of toluene with high selectivity and high yield.

According to still another embodiment, the alkali metal may be Li or Na.

According to still another embodiment, the catalyst may be a Y-zeolite catalyst having an FAU structure. The zeolite having an FAU structure includes a pore opening corresponding to a 12-oxygen ring, which is larger than the pore opening of an LTA structure having a pore opening corresponding to an 8-oxygen ring. Thus, such an FAU structure has a smaller spatial limitation during ion exchange.

According to still another embodiment, the Y-zeolite having an FAU structure may have a Si/Al molar ratio larger than 1.5 (Si/Al>1.5).

According to still another embodiment, the catalyst may be a metal halide catalyst.

According to still another embodiment, the catalyst may be a metal halide catalyst including a cation selected from transition metals and post-transition metals; and a halogen anion.

According to still another embodiment, the catalyst may be a metal chloride in view of the preparation of toluene with high selectivity and high yield.

According to yet another embodiment, the metal chloride may be AlCl₃, VCl₃ or CuCl₂.

In another aspect, there is provided a method for preparing toluene from 2-methylfuran by using the catalyst.

According to an embodiment, the method may include carrying out reaction between 2-methylfuran and ethylene in the presence of the catalyst and an organic solvent to obtain toluene through cycloaddition and dehydration.

According to another embodiment, the reaction temperature and reaction pressure are 200-300° C. and an initial ethylene pressure of 20-37 bars, respectively. As used herein, ‘initial ethylene pressure’ means the pressure when ethylene gas is injected. Particularly, the reaction temperature may be at least 200° C., at least 210° C., at least 220° C., at least 230° C., at least 240° C. or at least 250° C.; and at most 300° C., at most 290° C., at most 280° C., at most 270° C., at most 260° C. or at most 250° C. For example, the reaction temperature may be 240-260° C., or 250° C.

According to still another embodiment, the reaction time may be 4-48 hours. Particularly, the reaction time may be at least 4 hours, at least 6 hours, at least 8 hours, at least 10 hours, at least 12 hours, at least 14 hours, at least 16 hours, at least 18 hours or at least 20 hours; and at most 48 hours, at most 46 hours, at most 44 hours, at most 42 hours, at most 40 hours, at most 38 hours, at most 36 hours, at most 34 hours, at most 32 hours, at most 30 hours, at most 28 hours, at most 26 hours or at most 24 hours. For example, the reaction time may be 6-24 hours, or 10-30 hours.

According to still another embodiment, the organic solvent may be a polar aprotic organic solvent so that side reactions may be inhibited during the preparation of toluene and thus toluene may be obtained with high yield and high selectivity.

According to yet another embodiment, the polar aprotic organic solvent may be at least one selected from the group consisting of ethyl acetate, acetone, dimethylformamide, acetonitrile, dimethyl sulfoxide, 1,4-dioxane and tetrahydrofuran. Particularly, the polar aprotic organic solvent may be at least one selected from 1,4-dioxane and tetrahydrofuran in view of toluene production efficiency.

The examples and experiments will now be described. It is apparent to those skilled in the art that the following examples and experiments are for illustrative purposes only and not intended to limit the scope of the present disclosure.

### Comparative Examples 1-6

Herein, as solid acid catalysts with a silica-alumina composition, zeolite H-Beta (available from Zeolyst International, Comparative Example 1) including micropores and having properties as a Bronstead acid, H—Y (available from Zeolyst International, Comparative Example 2) and a silica-alumina aerogel catalyst (SAA-57, Comparative Example 3) including mesopores were used. In addition, Na cation-exchanged Na-Beta zeolite catalyst (Comparative Example 4) was used. In the case of the Na-Beta catalyst, it was obtained by dissolving NH₄-Beta zeolite (available from Zeolyst International) in 0.5 M aqueous sodium hydrogen carbonate solution and carrying out ion exchange by agitation at room temperature for 24 hours. Further, Sn-Beta (Comparative Example 5) and Zr-Beta catalyst (Comparative Example 6) were prepared and each catalyst had properties as a Lewis acid through the direct substitution of the zeolite BETA backbone with a metal.

### Examples 1-7

Na—Y (CBV100, SiO₂/A1203=5.1) and Na—X (SiO₂/A1203=2) were obtained from Zeolyst and Tosoh Corporation and used as base precursors. To carry out ion exchange, 1 g of Na—Y catalyst was dissolved in 0.1 M aqueous metal nitrate or metal chloride solution, and agitated thoroughly at 80° C. for 24 hours. Then, the solid was filtered and separated from the liquid material, dried at 100° C. for 12 hours, and sintered at 550° C. for 6 hours to obtain catalysts. In the case of the Na—Y catalyst, it was dissolved in 0.05 M aqueous sodium hydrogen carbonate solution for washing before the reaction, and agitated at room temperature for 1 hour. After the filtering, it was subjected to drying and calcination as mentioned above and used for the reaction. Depending on the exchanged metal ion, the catalysts were designated as Li—Y (Example 1), Na—Y (Example 2), K—Y (Example 3), Cs—Y (Example 4), Cu—Y (Example 5), Zn—Y (Example 6) and Na—X (Example 7).

### Test Example 1. Preparation of Toluene Using Catalysts of Comparative Examples 1-3

To convert 2-methylfuran into toluene, a 150 mL autoclave having an impeller attached thereto was used. Each of the catalysts according to Comparative Examples 1-3 was introduced in an amount of 0.15 g, and then 1.07 M 2-methylfuran (99% purity, Sigma Aldrich) mixed with 30 mL of 1,4-dioxane as a solvent was introduced to the reactor. Then, nitrogen was used to discharge the air contained in the reactor, and the reactor was filled with ethylene gas to 30 bars. After that, the impeller was operated under 300 rpm to carry out agitation, while increasing the reaction temperature to 250° C. After the reaction mixture arrives at the reaction temperature, the reaction was maintained for 8 hours, and the reaction product was analyzed by GC. The results were shown in the following Table 1.

TABLE 1

| | X_{MF} | Y_{TOL} | S_{TOL} | S_{AT} | S_{CI} | S_{AC} | S_{BF} | S_{OLI} |
|---|---|---|---|---|---|---|---|---|
| Catalyst | (%) | (%) | (%) | (%) | (%) | (%) | (%) | (%) |
| H-Beta | 93.79 | 25.21 | 26.88 | 7.34 | 10.30 | 0.71 | 0.47 | 4.78 |
| H-Y | 85.57 | 14.16 | 16.54 | 14.75 | 16.36 | 0.61 | 0.29 | 9.23 |
| SAA-57 | 97.33 | 18.49 | 19.00 | 4.09 | 5.66 | 0.00 | 0.42 | 8.43 |

(X_{MF} = conversion, Y_{TOL} = yield of toluene, S_{TOL} = Toluene selectivity, S_{AT} = Alkylated toluene selectivity, S_{CI} = ethylene-methylfuran cycloaddition isomer selectivity, S_{AC} = Alkylated cyclization by-product selectivity, S_{BF} = Benzofuran selectivity, S_{OLI} = methylfuran oligomer by-product selectivity)

After the test, all of the catalysts according to Comparative Examples 1-3, which exhibit excellent activity in the preparation of para-xylene, showed significantly low toluene selectivity (19-27%). In addition, it was shown that side-reaction products, such as oligomers (OLI), alkyl group-added by-products (AT) and cyclized by-products (CI, AC), were produced in a large amount.

### Test Example 2. Preparation of Toluene Using Catalysts of Examples 1-6

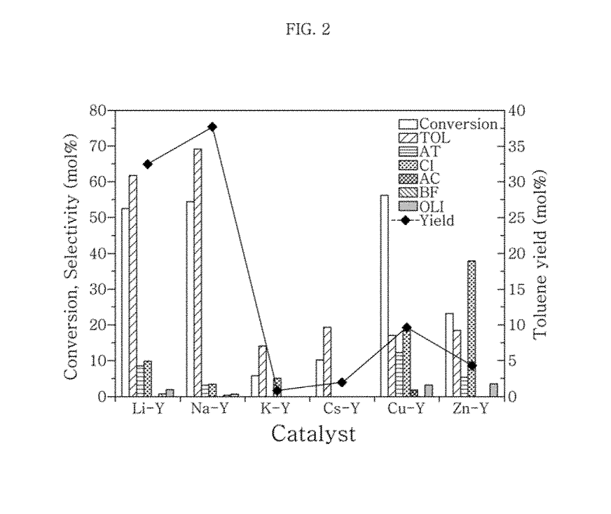

Toluene was prepared by using the catalysts obtained from Examples 1-6 according to the same method as Test Example 1. The test results were shown in FIG. 2.

After the test, it was shown that Li-exchanged catalyst (Example 1) and Na-exchanged catalyst (Example 2) among the metal ion-exchanged zeolite catalysts provided a high selectivity of 60-70% in the preparation of toluene. Such catalysts provided a conversion of about 50%, which is lower than the conversion (85-97%) provided by the Bronstead acid catalysts according to Comparative Examples 1-3. However, side reactions were inhibited so that the toluene selectivity was increased. The reason for such a low conversion is that the ion-exchanged Y-zeolites have properties as Lewis acids so that dehydration proceeds slowly. However, their properties as Lewis acids inhibit other side reactions to increase the toluene selectivity.

### Test Example 3. Preparation of Toluene Using Catalyst of Example 2

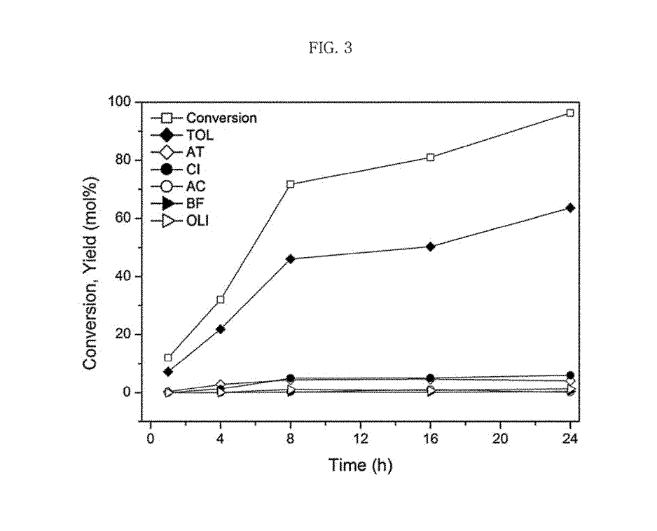

In the same manner as Test Example 1, reaction was carried out by using the catalyst of Example 2 under an initial ethylene gas pressure of 35 bars for 24 hours. The test results were shown in FIG. 3.

After the test, it can be seen that methylfuran is converted continuously into toluene as the reaction time increases. Finally, after carrying out the reaction for 24 hours, a conversion of methylfuran reached to 96% and yield of toluene reached to 65%.

### Test Example 4. Analysis for Surface Composition and Specific Surface Area/Pore Size of Catalysts of Examples 1-6

The catalysts of Examples 2-6 were analyzed for metal ion exchange degrees by scanning electron microscopy (SEM)/energy dispersive X-ray spectroscopy (EDX). The results were shown in the following Table 2. According to the results of EDX analysis, a different kind of metal provided a different ion exchange degree. Particularly, K and Zn provided a higher ion exchange degree as compared to Cs and Cu.

In addition, nitrogen adsorption analysis was carried out to determine the specific surface area and pore volume of each catalyst and the results were shown in the following Table 3. Each of the catalysts of Examples 1 and 2 showed the highest micropore volume and specific surface area. It is though that this is because the ion size of Li metal and that of Na metal are smaller as compared to the other metals, and thus pore clogging caused by metal ions is decreased.

TABLE 2

| | | EDX atomic % | | | | | | | |
|---|---|---|---|---|---|---|---|---|---|
| | Si/Al^{a} | Si | Al | Na | K | Cs | Cu | Zn | Si/Al^{b} |
| Ex. 2 | 2.55 | 65.83 | 20.60 | 13.56 | — | — | — | — | 3.20 |
| Ex. 3 | — | 64.16 | 19.59 | 2.47 | 13.78 | — | — | — | 3.28 |
| Ex. 4 | — | 60.16 | 21.24 | 6.41 | — | 12.20 | — | — | 2.83 |
| Ex. 5 | — | 63.56 | 21.44 | 5.70 | — | — | 9.29 | — | 2.96 |
| Ex. 6 | — | 63.22 | 21.51 | 0.00 | — | — | — | 15.28 | 2.94 |

^{a}Si/Al ratio in the parent material, Na-Y based on ICP
^{b}Si/Al ratio in the resulting alkali-exchanged zeolites based on EDX measurement

TABLE 3

| | | | | | | Total acid |
|---|---|---|---|---|---|---|
| | S_{BET}^{a} | S_{micro}^{b} | Sₑₓₜₑᵣₙₐₗ^{c} | Vₚₒᵣₑ^{d} | V_{micro}^{e} | sites^{f} |
| | (m²g⁻¹) | (m²g⁻¹) | (m²g⁻¹) | (cm³g⁻¹) | (cm³g⁻¹) | (mmol · g⁻¹) |
| Ex. 1 | 688 | 675 | 13 | 0.391 | 0.351 | 1.068 |
| Ex. 2 | 684 | 665 | 19 | 0.398 | 0.350 | 0.997 |
| Ex. 3 | 617 | 598 | 19 | 0.332 | 0.313 | 0.337 |
| Ex. 4 | 460 | 456 | 4 | 0.262 | 0.236 | 0.392 |
| Ex. 5 | 624 | 594 | 29 | 0.342 | 0.316 | 1.807 |
| Ex. 6 | 536 | 513 | 23 | 0.289 | 0.273 | 0.913 |

^{a}BET surface area
^{b}Micropore area obtained by t-plot analysis
^{c}External surface area obtained by t-plot analysis combined with BET surface area
^{d}Single point adsorption total pore volume (P/P₀ = 0.99)
^{e}Single point adsorption micropore volume (P/P₀ = 0.20)
^{f}Total acid sites were measured by NH₃-TPD.

### Test Example 5. Preparation of Toluene Using Catalysts of Example 7 and Comparative Examples 4-6

In the same manner as Test Example 1, reaction was carried out by using the catalysts of Example 7 and Comparative Examples 4-6 for 8 hours. The test results were shown in the following Table 4.

After the test, it was shown that both Example 7 (Na—X) and Comparative Example 4 (Na-Beta) exchanged with Na cation similarly to the catalyst of Example 2 and having a different zeolite backbone structure showed a significantly low methylfuran conversion and toluene selectivity, as compared to the catalyst of Example 2. Particularly, even though reaction was carried out by using the Na-Beta catalyst for 24 hours, a significantly lower methylfuran conversion and toluene selectivity were obtained. In addition, Sn-Beta (Comparative Example 5) and Zr-Beta (Comparative Example 6) substituted with a metal directly on the zeolite BETA backbone and having properties as Lewis acids showed a higher methylfuran conversion and toluene selectivity as compared to Na-Beta (Comparative Example 4), but still provided a lower toluene selectivity as compared to Na—Y (Example 2).

TABLE 4

| | X | Y_{TOL} | S_{TOL} | S_{AT} | S_{CI} | S_{AC} | S_{BF} | S_{OLI} |
|---|---|---|---|---|---|---|---|---|
| Catalyst | (%) | (%) | (%) | (%) | (%) | (%) | (%) | (%) |
| Na-Y | 54.52 | 37.73 | 69.20 | 3.09 | 3.45 | 0.00 | 0.23 | 0.50 |
| Na-X | 4.55 | 0.98 | 21.61 | 0.87 | 0.00 | 0.00 | 0.00 | 0.00 |
| Na- | 19.58 | 2.80 | 14.31 | 2.80 | 3.50 | 0.00 | 0.00 | 8.27 |
| Beta* | | | | | | | | |
| Sn-Beta | 29.53 | 4.26 | 14.42 | 14.48 | 25.87 | 2.58 | 0.54 | 5.98 |
| Zr-Beta | 75.75 | 14.11 | 18.62 | 5.97 | 36.33 | 2.05 | 0.55 | 1.67 |

Na-Beta*: Results of reaction for 24 hours

### Test Example 6. Preparation of Toluene Using Metal Chloride Catalysts

In the same manner as Test Example 1, various metal chloride catalysts including AlCl₃, CrCl₃, ZnCl₂, SnCl₄, YbCl₃, VCl₃, InCl₃, MgCl₂, LaCl₃, NiCl₂ and CuCl₂ were used to carry out reaction for 8 hours. The test results were shown in the following Table 5.

After the test, it was shown that a metal chloride catalyst having properties as a Lewis acid also provided high activity in the preparation of toluene. Among such different metal chlorides, AlCl₃ showed the highest yield of toluene (45%), which was better as compared to the catalyst of Example 2 (38%). When the reaction time was increased to 24 hours, AlCl₃ showed a methylfuran conversion of 99% and yield of toluene of 70%. Thus, it could be seen that metal chlorides showed excellent performance in the preparation of toluene.

TABLE 5

| | X_{MF} | Y_{TOL} | S_{TOL} | S_{AT} | S_{CI} | S_{AC} | S_{BF} | S_{OLI} |
|---|---|---|---|---|---|---|---|---|
| Catalyst | (%) | (%) | (%) | (%) | (%) | (%) | (%) | (%) |
| AlCl₃ | 82.24 | 45.19 | 54.95 | 5.63 | 9.47 | 0.90 | 0.33 | 3.57 |
| AlCl₃* | 99.20 | 69.63 | 70.20 | 0.56 | 0.87 | 0.00 | 0.18 | 3.40 |
| VCl₃ | 81.67 | 41.61 | 50.95 | 3.15 | 5.10 | 1.15 | 0.00 | 6.04 |
| CrCl₃ | 76.24 | 36.62 | 48.04 | 1.10 | 2.20 | 1.44 | 1.26 | 3.96 |
| SnCl₄ | 91.83 | 31.36 | 34.15 | 5.36 | 7.80 | 0.24 | 0.00 | 4.65 |
| YbCl₃ | 74.29 | 27.51 | 37.04 | 5.28 | 7.54 | 0.55 | 0.00 | 5.09 |
| InCl₃ | 93.66 | 11.08 | 11.83 | 9.05 | 10.65 | 0.00 | 0.00 | 11.57 |
| LaCl₃ | 11.41 | 2.58 | 22.58 | 0.15 | 0.79 | 1.50 | 0.00 | 1.83 |
| CuCl₂ | 79.90 | 41.26 | 51.64 | 5.48 | 7.60 | 0.65 | 0.00 | 2.37 |
| ZnCl₂ | 91.70 | 30.27 | 33.01 | 5.17 | 6.17 | 0.54 | 0.00 | 4.97 |
| MgCl₂ | 14.92 | 3.51 | 23.53 | 1.90 | 4.00 | 1.59 | 0.00 | 3.50 |
| NiCl₂ | 15.17 | 2.90 | 19.12 | 0.53 | 0.89 | 2.52 | 0.00 | 4.53 |

AlCl₃*: Results of reaction for 24 hours

### Test Example 7. Preparation of Toluene Using Metal Triflate Catalysts

Metal triflate means a compound in which a metal cation and triflate (OTf) anion are present in the form of salt. In the same manner as Test example 1, various metal triflates including Sc(OTf)₃, In(OTf)₃ and Cu(OTf)₃ were used as catalysts to carry out reaction for 8 hours. The results were shown in the following Table 6.

After the test, a non-metal anion bound with a metal cation was found to have a significant effect upon catalytic activity. Triflate catalysts showed significantly lower toluene selectivity as compared to the halide (chloride) catalysts. It could be also seen that the resultant liquid product had a black color, which suggests that furan oligomerization was accelerated. Therefore, selection of an adequate non-metal anion in a metal catalyst is very important for increasing the activity of a catalyst for preparing toluene.

TABLE 6

| | X_{MF} | Y_{TOL} | S_{TOL} | S_{AT} | S_{CI} | S_{AC} | S_{BF} | S_{OLI} |
|---|---|---|---|---|---|---|---|---|
| Catalyst | (%) | (%) | (%) | (%) | (%) | (%) | (%) | (%) |
| Sc(OTf)₃ | 98.41 | 10.68 | 10.85 | 0 | 0 | 0.3 | 3.84 | 2.86 |
| In(OTf)₃ | 99.26 | 8.69 | 8.75 | 0 | 0 | 0.61 | 3.96 | 2.3 |
| Cu(OTf)₃ | 99.44 | 1.74 | 1.75 | 0 | 0 | 0.68 | 1.32 | 2.9 |

### Test Example 8. Results of Toluene Preparation Using Various Organic Solvents

In the same manner as Test Example 1, the catalyst of Example 2 was used to carry out reaction of 4 hours in the presence of various organic solvents. The test results were shown in the following Table 7.

TABLE 7

| | | | | | Rate | |
|---|---|---|---|---|---|---|
| | | X_{MF} | S_{TOL} | TOF | (mmol · | C. B. |
| Solvent | Polarity | (%) | (%) | (h⁻¹) | g⁻¹ · h⁻¹) | (%) |
| n-Heptane | 0.1 | 30.15 | 6.72 | 0.77 | 0.77 | 73.49 |
| o-Xylene | 2.5 | 14.97 | 3.83 | 0.21 | 0.21 | 92.48 |
| 1,4-Dioxane | 4.8 | 31.97 | 68.33 | 7.73 | 7.71 | 94.39 |
| DMSO | 7.2 | Solvent decomposed, and the | | | | |
| | | solution was solidified. | | | | |
| Water | 10.2 | 59.57 | 9.98 | 1.98 | 1.97 | 49.51 |
| Neat | — | 0.39 | 7.71 | 0.09 | 0.09 | 99.64 |
| condition | | | | | | |

After the test, it could be seen that use of 1,4-dioxane solvent as a polar solvent provided a significantly higher toluene selectivity as compared to n-heptane or o-xylene as a non-polar solvent. In addition, when no solvent was used (neat condition), little conversion of methylfuran occurred (conversion of 0.39%). Further, in the case of DMSO having higher polarity as compared to 1,4-dioxane, it was decomposed under the reaction condition. In the case of water as a polar aprotic solvent, a high methylfuran conversion was obtained but a significantly low toluene selectivity was provided.

While the exemplary embodiments have been shown and described, it will be understood by those skilled in the art that various changes in form and details may be made thereto without departing from the spirit and scope of the present disclosure as defined by the appended claims. Therefore, it is intended that the present disclosure not be limited to the particular exemplary embodiments disclosed as the best mode contemplated for carrying out the present disclosure, but that the present disclosure will include all embodiments falling within the scope of the appended claims.

What is claimed is:

1. A catalyst for use in the preparation of toluene from 2-methylfuran,
wherein the catalyst is a Lewis acid catalyst; and
wherein the catalyst is a zeolite catalyst ion-exchanged with at least one metal.

2. The catalyst for use in the preparation of toluene according to claim 1, wherein the zeolite catalyst is ion-exchanged with at least one metal selected from the group consisting of alkali metals, transition metals and post-transition metals.

3. The catalyst for use in the preparation of toluene according to claim 2, wherein the zeolite catalyst is ion-exchanged with at least one alkali metal.

4. The catalyst for use in the preparation of toluene according to claim 3, wherein the zeolite catalyst is ion-exchanged with Li or Na.

5. The catalyst for use in the preparation of toluene according to claim 1, wherein the zeolite catalyst is a Y-zeolite catalyst having an FAU structure.

6. A catalyst for use in the preparation of toluene from 2-methylfuran,
wherein the catalyst is a Lewis acid catalyst; and
wherein the catalyst is a metal halide catalyst.

7. The catalyst for use in the preparation of toluene according to claim 6, wherein the metal halide catalyst comprises: at least one cation selected from the group consisting of transition metals and post-transition metals; and at least one halogen anion.

8. The catalyst for use in the preparation of toluene according to claim 7, wherein the metal halide catalyst is a metal chloride.

9. The catalyst for use in the preparation of toluene according to claim 8, wherein the metal halide catalyst is AlCl₃, VCl₃ or CuCl₂.

10. A method for preparing toluene from 2-methylfuran, the method comprising carrying out reaction of 2-methylfuran with ethylene in the presence of the Lewis acid catalyst according to (a) or (b) below and an organic solvent:
(a) a catalyst for use in the preparation of toluene from 2-methylfuran, wherein the catalyst is a zeolite catalyst ion-exchanged with at least one metal,
(b) a catalyst for use in the preparation of toluene from 2-methylfuran, wherein the catalyst is a metal halide catalyst.

11. The method for preparing toluene according to claim 10, wherein the reaction temperature and reaction pressure are 200-300° C. and an initial ethylene pressure of 20-37 bars, respectively.

12. The method for preparing toluene according to claim 10, wherein the reaction time is 4-48 hours.

13. The method for preparing toluene according to claim 10, wherein the organic solvent is a polar aprotic organic solvent.

14. The method for preparing toluene according to claim 13, wherein the polar aprotic organic solvent is at least one selected from the group consisting of 1,4-dioxane and tetrahydrofuran.
